(19) United States
(12) Patent Application Publication
(10) Pub. No.: US 2020/0030590 A1
(43) Pub. Date: Jan. 30, 2020

(54) TRANSDERMAL DRUG DELIVERY DEVICES AND METHODS

(71) Applicant: CHRONO THERAPEUTICS INC., Hayward, CA (US)

(72) Inventors: Daniela Tamar BUCHMAN, Mountain View, CA (US); Ling-Kang TONG, Hayward, CA (US); Michael Thomas WRIGHT, Allen, TX (US); Adam Blake HARTMAN, Denton, TX (US)

(21) Appl. No.: 16/484,477

(22) PCT Filed: Feb. 13, 2018
(86) PCT No.: PCT/US2018/018047

§ 371 Date: Aug. 8, 2019

Related U.S. Application Data
(60) Provisional application No. 62/458,325, filed on Feb. 13, 2017.

(51) Int. Cl. A61M 35/00

(52) U.S. Cl. CPC A61M 35/003; A61M 2205/128; A61M 2205/106; A61M 2209/06

(57) ABSTRACT

A formulation delivery system includes a cartridge, a control unit, and a stiction breaking element. The cartridge includes a reservoir chamber, a bolus chamber, a reservoir chamber piston, and a bolus chamber piston. The reservoir chamber piston is configured to move within the reservoir chamber to expel formulation from the reservoir chamber into the bolus chamber. The bolus chamber piston is configured to move within the bolus chamber to expel the formulation from the bolus chamber to a patient. The control unit is configured to engage with the cartridge and includes a control configured to activate the reservoir chamber piston or the bolus chamber piston. The piston stiction breaking element is configured to break a stiction between the reservoir chamber piston and the reservoir chamber or between the bolus chamber piston and the bolus chamber.

### CROSS REFERENCE TO RELATED APPLICATIONS

This application claims priority to U.S. Provisional Application No. 62/458,325, filed Feb. 13, 2017, titled “TRANSDERMAL DRUG DELIVERY DEVICES AND METHODS”, the entirety of which is incorporated by reference herein.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present application relates generally to devices and methods for providing a bioactive agent to a user.

### BACKGROUND

Medicinal drugs are given to people to manage or improve their health for a variety of reasons, such as to prevent or treat a medical condition or disease such as diabetes, Parkinson's disease, ulcerative colitis, or to manage nicotine or another addiction or dependency, or to manage pain.

Some medicinal drugs are rapidly metabolized by the body. Multiple doses of the drug over a period of time are therefore often needed to provide a desired effect. In addition to having desired preventative or therapeutic effects, medicinal drugs can also have negative side-effects on the body that can range from irritating to life-threatening. A person's body can also develop tolerance to a drug and experience a diminished response to the drug after taking it for a period of time and require higher doses to have an effect, resulting in increased drug use and additional side-effects. Despite their negative side-effects, a person generally takes a medicinal drug because, on the whole, the drug causes more good than harm. It is beneficial to a person taking a drug, however, to minimize the amount of drug taken to prevent or minimize tolerance and other unwanted side-effects while still receiving the desired therapeutic effect from the drug.

Tobacco use, such as smoking, causes serious health problems and can lead to premature death. According to the United States Center for Disease Control (CDC), tobacco use causes more than 5 million deaths per year as well as contributing to the development of serious illnesses such as cancer, diabetes, heart disease, lung disease (bronchitis, chronic airway destruction, emphysema), and stroke. Despite anti-smoking advertising campaigns, legislation, taxation, and development of smoking cessation products to stop or prevent people from using tobacco, tobacco sales remains a multibillion dollar industry, generating an estimated $35 billion dollars per year in profits. Further, it is difficult for a person to stop using a tobacco product because tobacco contains nicotine. Nicotine is highly addictive, and not having the nicotine causes harsh withdrawal symptoms. It is very difficult for a person to overcome a nicotine addiction and stop smoking.

Medicinal drugs can be taken by tobacco users to help them to overcome their nicotine addiction and stop using tobacco. Some products to help a person stop smoking contain small amounts of nicotine as a medicinal drug to minimize withdrawal symptoms and gradually wean a person from their nicotine addiction. Medicinal smoking cessation drugs, such as nicotine, have to be taken over an extended period of time (often over the course of many months) to give the body time to adjust to having less nicotine. Medicinal drugs, medical devices and other products, including smoking cessation products, are regulated in the United States by the U.S. Food and Drug Administration (FDA). FDA approved products on the market to help a person quit smoking include various medicinal drugs that require a doctor's prescription as well as over-the-counter products. These products include capsules or tablets, gums, inhalers, lozenges, nasal sprays, and skin patches. These products have thus far been inadequate to get people to stop smoking: 68.9% of adult cigarette smokers say they want to stop smoking, and every year some 42.7% make an attempt to stop smoking, but are unsuccessful.

Existing smoking cessation products and other therapeutic and prophylactic treatments for health issues suffer from a variety of problems. They may be inconvenient or socially awkward to use. They may require careful and troublesome tracking of when they were used and how much was used to prevent overdosing. They may act too slowly after being administered and not produce a desired effect when it's needed. They may not be readily available when they are needed (such as while a person is sleeping). None have been wholly effective to for preventing or treating various medical or other conditions. Smoking, for example, remains a significant health and social problem.

Further, in some drug delivery systems, such as smoking cessation delivery systems, pistons can be used to exert a force on a drug formulation contained therein to deliver the formulation to the patient. In many cases, however, particularly where the delivery system has sitting for a long time at a warehouse or on a shelf, the piston(s) can become stuck or difficult to move. This phenomena can be referred to as stiction. Stiction can, in some cases, increase with the amount of time the device spends in storage. A force is often therefore required to be applied to the piston to break the stiction between the piston and the chamber the piston sits in (e.g., between the piston and a reservoir chamber that holds formulation). Depending on the storage conditions and the solution within the dosing/bolus chamber or drug reservoir chamber, the force needed to break the stiction can be quite large. Further, if the system relies on one of the springs that engages with the pistons to break the stiction, then the required stiffness of the spring can be quite large. The use of a stiffer spring can undesirably increase the size of the device, costs of the device, the force the user needs to exert to assemble the device, and result in less desirable user experience. In addition, after the stiction is broken, the force needed from the springs to move the piston(s) is much lower than the force required to break the stiction, resulting in over-sized springs and/or forces during the normal course of use.

What is needed are new and improved systems, devices and methods for delivering drugs and other bioactive agents, such as smoking cessation agents, that overcome some of these deficiencies.

### SUMMARY OF THE DISCLOSURE

The present invention relates generally to systems for delivering bioactive agents and methods for using the systems to deliver bioactive agents.

In general, in one embodiment, a formulation delivery system includes a cartridge, a control unit, and a stiction breaking element. The cartridge includes a reservoir chamber, a bolus chamber, a reservoir chamber piston, and a bolus chamber piston. The reservoir chamber is configured to hold a formulation therein. The bolus chamber is in fluid communication with the reservoir chamber through a fluid communication pathway and is configured to hold a portion of the formulation from the reservoir chamber. The reservoir chamber piston is configured to move within the reservoir chamber to expel the formulation from the reservoir chamber into the bolus chamber. The bolus chamber piston is configured to move within the bolus chamber to expel the formulation from the bolus chamber to a patient. The control unit is configured to engage with the cartridge and includes a control configured to activate the reservoir chamber piston or the bolus chamber piston. The piston stiction breaking element is configured to break a first stiction between the reservoir chamber piston and the reservoir chamber or between the bolus chamber piston and the bolus chamber.

This and other embodiments can include one or more of the following features. The piston stiction breaking element can be configured such that, as the cartridge and the control unit are engaged, the piston stiction breaking element applies a force to the reservoir chamber piston or the bolus chamber piston to break the first stiction. The fluid communication pathway can be configured such that, when the piston stiction breaking element applies the force to the reservoir chamber piston or the bolus chamber piston to break the first stiction, the force is transferred through the formulation to the other of the reservoir chamber piston or the bolus chamber piston to break a second stiction. The reservoir chamber piston and the bolus chamber piston both move by a distance d when the first and second stictions are broken. The distance d can be between approximately 0.5 mm and 2.5 mm. The control unit can further include a first shaft and first spring, and the first spring can be configured to provide force to the first shaft to move the reservoir chamber piston or the bolus chamber piston. The piston stiction breaking element can be an extension on the first shaft, and the extension can be configured such that it pushes the reservoir chamber piston or bolus chamber piston as the cartridge and control unit are engaged to break the first stiction. The control unit can include a stop therein, and the stop can be configured to engage with the first spring or the first shaft as the control unit and cartridge are engaged to prevent movement of the first spring and the first shaft distally and to allow the extension to push the reservoir chamber piston or bolus chamber piston. The extension can be configured such that the extension pushes the reservoir chamber piston. The first spring can be configured to provide approximately 15N of force or less to break the first stiction. The piston stiction breaking element can be a tab configured to engage with the reservoir chamber piston or the bolus chamber piston. The tab can be a breakaway tab. The breakaway tab can be configured such that a force applied to break off the breakaway tab also breaks the first stiction. The tab can be attached to or part of a packaging of the cartridge, and removing the packaging can cause the tab to break the first stiction. The piston stiction breaking element can be part of a spring system, and the spring system can include a first spring having greater stiffness than a second spring. The first spring can be configured to provide force to the bolus chamber piston or the reservoir chamber piston to break the first stiction. The first spring can include a first portion and a second portion, and the first portion can have a higher stiffness than the second portion. The piston stiction breaking element can be a rod extending from the cartridge, and the rod can be configured to push the reservoir chamber piston or the bolus chamber piston as the cartridge and the control unit are engaged. The drug delivery system can be a transdermal drug delivery system that includes a transdermal membrane in fluid connection with the bolus chamber. The formulation delivery system can further include a control valve with a first position forming the first fluid communication pathway between the bolus chamber and the reservoir chamber and a second position forming a second fluid communication pathway between the bolus chamber and a transdermal membrane. A total force required to start movement of the bolus chamber piston or reservoir chamber piston can be between approximately 2N and 15N.

In general, in one embodiment, a formulation delivery system includes a cartridge, a control unit, and a piston stiction breaking element. The cartridge includes a reservoir chamber configured to hold a formulation therein and a reservoir chamber piston configured to move within the reservoir chamber to expel the formulation from the reservoir chamber. The control unit is configured to engage with the cartridge and includes a shaft, a spring, and a control configured to activate the spring. The spring is configured to provide force to the shaft to move the reservoir chamber piston. The piston stiction breaking element is configured to break a first stiction between the reservoir chamber piston and the reservoir chamber.

This and other embodiments can include one or more of the following features. The formulation delivery system can further include a bolus chamber in fluid communication with the reservoir chamber through a fluid communication pathway. The bolus chamber can be configured to hold a portion of the formulation from the reservoir chamber. The formulation delivery system can further include a bolus chamber piston configured to move within the bolus chamber to expel the formulation from the bolus chamber to a patient. The fluid communication pathway can be configured such that, when the piston stiction breaking element applies the force to the reservoir chamber piston to break the stiction, the force is transferred through the formulation to the bolus chamber piston to break a second stiction between the bolus chamber piston and the bolus chamber. The reservoir chamber piston and the bolus chamber piston can both move by a distance d when the first and second stictions are broken. The distance d can be between approximately 0.5 mm and 2.5 mm. The piston stiction breaking element can be configured such that, as the cartridge and the control unit are engaged, the piston stiction breaking element applies a force to the reservoir chamber piston to break the first stiction. The piston stiction breaking element can be an extension on the shaft, and the extension can be configured such that it pushes the reservoir chamber piston as the cartridge and control unit are engaged to break the first stiction. The control unit can includes a stop therein, and the stop can be configured to engage with the spring or the shaft as the control unit and cartridge are engaged to prevent movement of the spring and the shaft distally and to allow the extension to push the reservoir chamber piston. The spring can be configured to provide approximately 15N or less of force to break the first stiction. The piston stiction breaking element can be a tab configured to engage with the reservoir chamber piston. The tab can be a breakaway tab. The breakaway tab can be configured such that a force applied to break off the breakaway tab also breaks the first stiction. The tab can be attached to or part of a packaging of the cartridge, and removing the packaging can cause the tab to break the first stiction. The piston stiction breaking element can be part of the spring, and the spring can include a first portion and a second portion. The first portion can have a higher stiffness than the second portion. The piston stiction breaking element can be a rod extending from the cartridge, and the rod can be configured to push the reservoir chamber piston as the cartridge and the control unit are engaged. The drug delivery system can be a transdermal drug delivery system including a transdermal membrane in fluid connection with the reservoir. A total force required to start movement of the reservoir chamber piston can be between approximately 2N and 15N.

In general, in one embodiment, a method of using a transdermal drug delivery system includes engaging a cartridge of the transdermal drug delivery system with a control unit of the transdermal drug delivery system. The cartridge includes a reservoir chamber with a formulation therein, a bolus chamber with a formulation therein, a reservoir chamber piston, and a bolus chamber piston. The method further includes applying force to lock the cartridge and control unit together, where the application of force activates a stiction breaking mechanism to break a first stiction between the reservoir chamber piston and the reservoir chamber or a second stiction between the bolus chamber piston and the bolus chamber. Additionally, the method includes allowing the formulation to be delivered to a patient.

This and other embodiments can include one or more of the following features. The force applied to the lock the cartridge and control unit together can be between approximately 12N and 35N. The application of force can break the first stiction and the second stiction.

In general, in one embodiment, a method of using a transdermal drug delivery system includes engaging a cartridge of the transdermal drug delivery system with a control unit of the transdermal drug delivery system. The cartridge includes a reservoir chamber with a formulation therein, a bolus chamber with a formulation therein, a reservoir chamber piston, and a bolus chamber piston. The method further includes pulling a tab to break a first stiction between the reservoir chamber piston and the reservoir chamber or a second stiction between the bolus chamber piston and the bolus chamber, applying force to lock the cartridge and control unit together, and allowing the formulation to be delivered to a patient.

This and other embodiments can include one or more of the following features. Pulling the tab can break the first stiction and the second stiction. Pulling the tab can include unwrapping the cartridge from a packaging, the unwrapping pulling the tab. The tab can be fixed to the packaging.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a transdermal drug delivery device with a piston stiction breaking mechanism

FIGS. 2A-2B show a transdermal drug delivery device with a piston stiction breaking mechanism that includes a bump on the reservoir shaft. FIG. 2A is a top view and FIG. 2B is a side view showing the overlap distance d between the bump and the piston.

FIGS. 2C-2F show use of the transdermal drug delivery device with the same stiction break concept of FIGS. 2A-2B. FIGS. 2C and 2E show the device before the first and second parts are engaged while FIGS. 2D and 2F show the device after the two parts are engaged.

FIG. 3 shows a graph of force vs displacement for a piston sitting for 6 months and a piston sitting for 18 hours.

FIG. 4 shows a portion of a disposable part of a transdermal drug delivery device with a piston stiction breaking mechanism that includes a tab.

FIG. 5 shows a transdermal drug delivery system with a piston stiction breaking mechanism that includes a rod.

FIG. 6 shows a transdermal drug delivery system with a piston stiction breaking mechanism that includes a tab.

FIGS. 7A-7B illustrate an embodiment of a transdermal drug delivery device with a piston stiction breaking mechanism that includes springs with varying stiffnesses.

### DETAILED DESCRIPTION

Described herein are drug delivery devices, such as transdermal drug delivery devices, that include a stiction breaking mechanism. By using such a stiction breaking mechanism, smaller springs can be used in the device, thereby reducing size, force the user has to exert to assemble the device, and cost. Improved methods for breaking the stiction of the piston or minimizing the effects of piston stiction are also described herein.

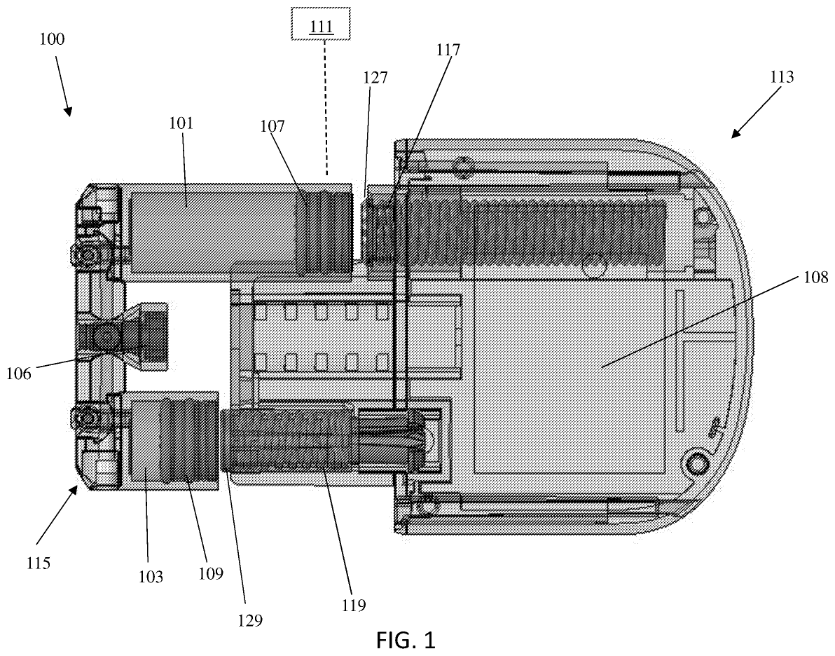

Referring to FIG. 1, a transdermal drug delivery device 100 can include a drug reservoir chamber 101 and a bolus (dosing) chamber 103 configured to deliver a formulation to the user, e.g., through a transdermal membrane. A reservoir chamber piston 107 can be positioned partially within reservoir chamber 101 while a bolus chamber piston 109 can be positioned partially within the bolus chamber 103. Springs 117, 119 can be connected to shafts 127, 129 so as to exert forces from the shafts 127, 129 to the pistons 107, 109. A control valve 106 positioned between the reservoir 101 and the bolus chamber 103 can have a first position that allows fluid communication from the reservoir 101 to the bolus chamber 103 and a second position allowing fluid communication from bolus chamber 103 and a transdermal membrane on the underside of the device 100. A control 108 (including electronics, a printed circuit board, a computer, a controller, and/or a motor) can activate delivery of the fluid. For example, the motor can activate the valve 106 to turn it in a first position to allow fluid flow between the reservoir chamber 101 and the bolus chamber 103 (thereby allowing the spring 117, shaft 127, and piston 107 to push fluid into the bolus chamber 103) and in a second position to allow fluid to flow out of the bolus chamber 103 (via force from the spring 119, shaft 129, and piston 109).

The delivery device can include a reusable part 113 and a disposable part 115. The disposable part 115 can include, for example, the control valve 106, bolus chamber 103, reservoir chamber 101, and pistons 107, 109 while the reusable part 113 can include the control 108, springs 117, 119, and shafts 127, 129.

The delivery device 100 can further include a stiction breaking mechanism 111 to break the stiction of the reservoir chamber piston 107 and/or the bolus chamber piston 109. The stiction breaking mechanism 111 can function to prepare the pistons 107, 109 for use in delivering fluid from the bolus chamber 109 to the patient. In some embodiments, the force exerted to break the stiction on one piston, such as reservoir chamber piston 107, can increase the pressure on the fluid in the reservoir chamber 101, which, because they are connected by the fluidic path, can increase pressure on the fluid in bolus chamber 103 to exert a force on the other piston, such as piston 109, so as to break the stiction on the second piston 109 as well.

In some embodiments, the piston stiction breaking mechanism 111 can be a rigid component of the reusable part 113. The rigid component can exert a force on the piston(s) 107, 109 when the reusable part is engaged with the disposable part to provide the necessary force to break the stiction. The rigid component can have a shape with a projection, notch, or other structure that can exert a force on the piston(s) 107, 109 without interfering with the engagement of the shafts 127, 129 with the piston. In some embodiments, the spring(s) 117, 119 can be configured to allow a portion of the rigid component to pass by the profile of the spring(s) 117, 119 to initially engage with the piston(s) 107, 109 to break the stiction. After the stiction is broken, the spring(s) 117, 119 can be activated to allow the shaft(s) 127, 129 to engage with the piston(s) 107, 109 and exert the force on the piston(s) 107, 109 as desired for the normal operation of the transdermal drug delivery device.

In some embodiments, a portion of the disposable part 115 or packaging of the disposable part 115 can be arranged in such a way that, when the user removes the disposable part 115 from the packaging, a portion of the packaging “nudges” the reservoir chamber piston 107 and breaks the stiction on both the reservoir and bolus chamber pistons 107, 109. In one such example, the piston stiction breaking mechanism 111 could be a tab or breakaway tab. In some cases, the disposable part 115 can include a breakaway pull tab that pulls or pushes the piston 107 and/or 109 until it breaks the stiction of the piston/s followed by the tab breaking away.

In some embodiments, the piston stiction breaking mechanism 111 can include a piston engagement surface or bump that is pushed inwards when the reusable part 113 and disposable part 115 are engaged. For example, the piston engagement surface can be part of the reservoir and/or bolus shafts 127, 129 and can have a rigid configuration.

In some embodiments, the spring(s) 117, 119 can have a plurality of different stiffness or different stiffness properties along different portions of the axis of the spring. For example, the spring(s) 117, 119 can have a lower stiffness in the area adjacent to the piston engagement surface and a higher stiffness further away from the piston engagement surface or vice versa. The spring(s) 117, 119 can be designed to exert a relatively high force on the associated piston 107, 109 when the reusable part 113 and the disposable part 115 are engaged. When the rigid component or bump and spring(s) 117, 119 are compressed during the engagement of the disposable part 115 and the reusable part 113 to exert a large enough force on the piston(s) 107, 109 to break the stiction.

A number of different embodiments of piston stiction breaking mechanisms are provided and discussed in the examples below.

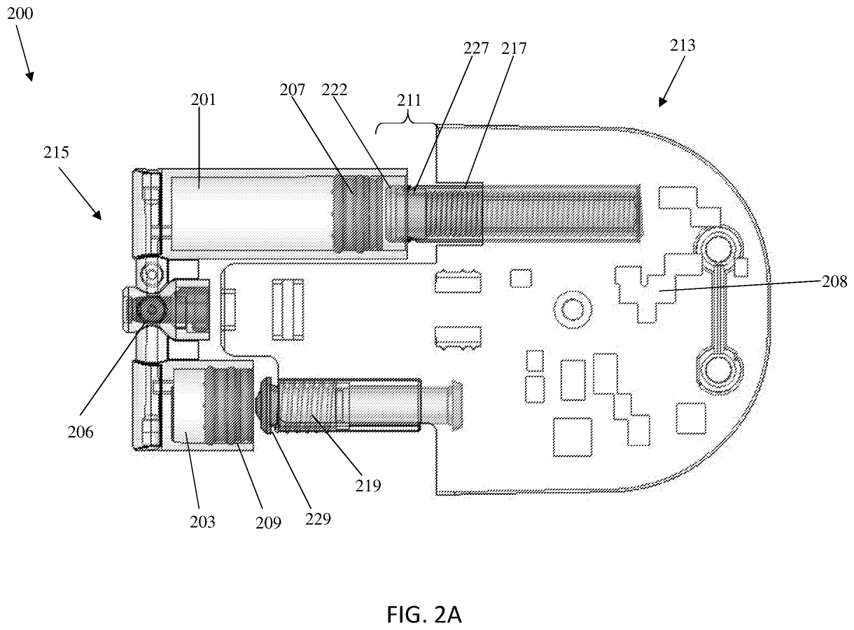

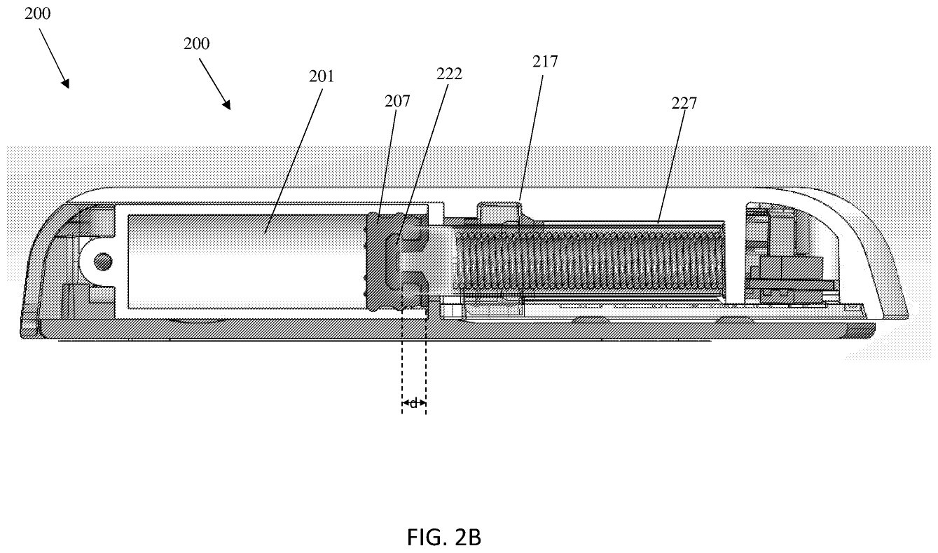

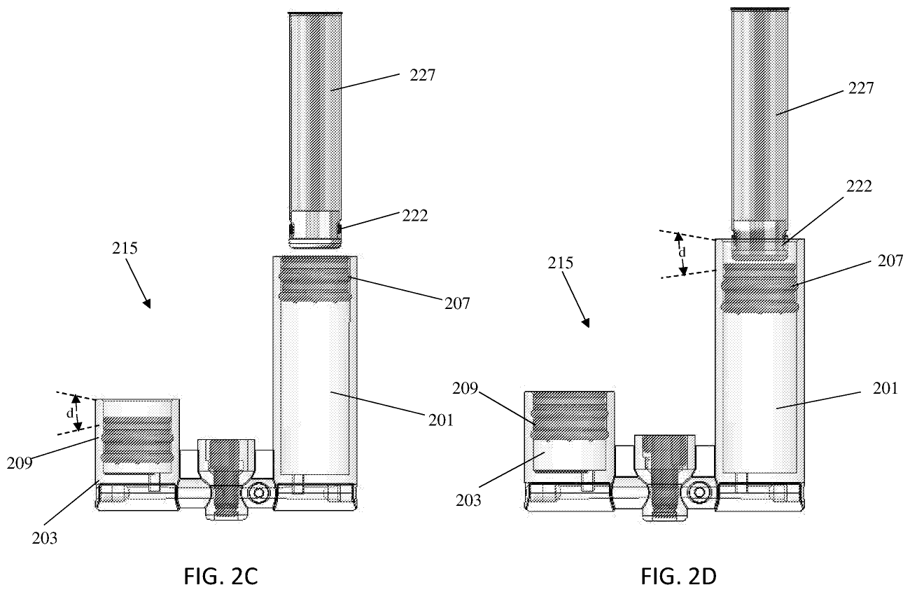

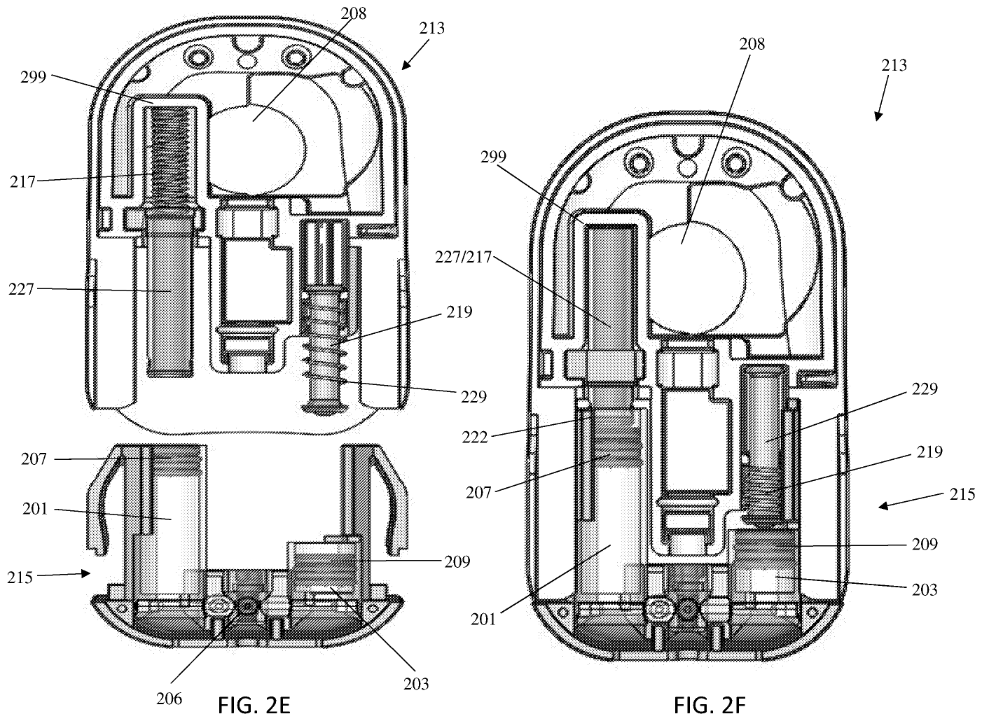

FIGS. 2A-2B illustrate an exemplary delivery device 200 including a reusable part 213 and a disposable part 215. The reusable part 213 includes a control 208, a reservoir shaft 227, a reservoir spring 217, a bolus shaft 229, and a bolus spring 219. The disposable part 215 includes a reservoir 201, a reservoir chamber piston 207, a bolus chamber 203, a bolus chamber piston 209, and a control valve 206. Additionally, the reservoir shaft 227 can include a piston stiction breaking mechanism 211 in the form of a bump 222 at the distal end thereof configured to engage with the reservoir chamber piston 207 as the reusable part 213 and disposable part 215 are engaged in order to break the stiction of the piston 207 (the bump 222 moves the piston 207 by overlap distance d). Because the fluid in the reservoir chamber 201 and bolus chamber 203 is incompressible, the breaking of the stiction 207 can place reverse pressure on the piston 209, thereby breaking the stiction of piston 209 as well.

FIGS. 2C-2F illustrate the use of a piston stiction breaking element 211. Prior to breaking the stiction (as shown in FIGS. 2C and 2E), the disposable part 215 can be disengaged from the reusable part 213 (and thus from the shaft 227/bump 222). The bolus chamber 203 can be only partially filled such that the bolus chamber piston 209 sits within the bolus chamber 203 offset from the end of the bolus chamber by a distance d. Distance d can be, for example, 0.5-2.5 mm, such as 1.5-2 mm. Further, the control valve 206 can be positioned so as to allow fluid to travel between the reservoir chamber 201 and the bolus chamber 203. Referring to FIGS. 2D and 2F, when the disposable part 215 and reusable part 213 are engaged, the shafts 227, 229 can move distally (via compression of the springs 217, 219) until the shaft 227 hits stop 299. The bump 222 can then push on the reservoir chamber piston 207, breaking its stiction and causing it to move within the reservoir chamber 201 by a distance d. Because the fluid in the reservoir chamber 201 and the bolus chamber 203 is not compressible, the force applied to the reservoir chamber piston 207 can cause fluid to move into the bolus chamber 203 and force the bolus chamber piston 209 to move backwards (by the distance d), thereby breaking the stiction on the bolus chamber piston 209 as well. Thus, when the stiction is broken on the reservoir chamber piston 207, the stiction on the bolus chamber piston 209 can also be broken.

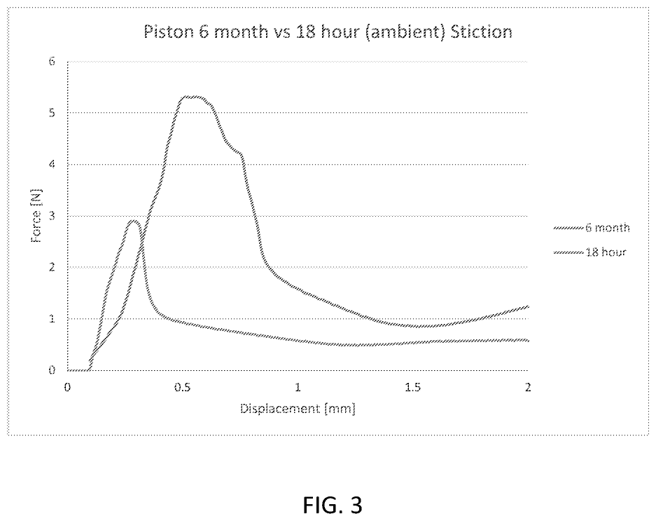

The piston stiction breaking mechanism 211 advantageously ensures that lower force is required to move pistons 207, 209 during normal use. That is, rather than being required to move pistons 207, 209 that have developed stiction while sitting on the shelf (e.g., for 6 months or more), the springs 217, 219 need only provide enough force to move pistons 207, 209 that have developed stiction during the course of use of the device (e.g., for 18 hours). A graph showing the amount of force required to displace a piston at 18 hours vs at 6 months is shown in FIG. 3. As shown, the amount of force required to break the stiction at 18 hours rather than 6 months decreases the force by almost half, e.g., to less than 3N. When used within system 200 (e.g., with springs 207 and 209), the force required by the reservoir spring 217 can be less than 15N, such as less than or equal to 10N while the force require by the bolus spring 219 can be less than 6N, such as less than 5N, less than 4N, or less than 3N. The total force required to start movement of the bolus and reservoir chamber pistons 209, 207 when using stiction breaking mechanism 211 can be between 2N-15N, such as 5N-10N while the force required to move fluid using the reservoir chamber piston 207 and bolus chamber piston 209 can be between 0.5 N/mm and 3 N/mm. Thus, using the stiction breaking mechanism 211 can decrease the total spring force required for system 200 by, for example, 40-50%.

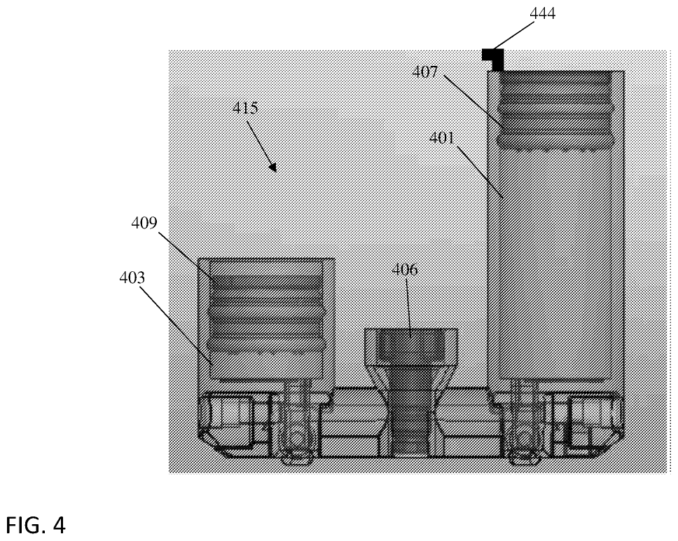

FIG. 4 illustrates a disposable part 415 of another exemplary delivery device. Similar to the other embodiments described herein, the disposable part 415 includes a reservoir chamber 401, a bolus chamber 403, a reservoir chamber piston 407, a bolus chamber piston 409, and a control valve 406. The disposable part 415 further includes a piston stiction breaking element in the form of a tab 444. The size, shape, and/or placement of the tab 444 can be such that the breaking off or removing the tab 444 requires a force that is greater than the force needed to break the stiction on the piston in the drug reservoir. In one embodiment, the user of the device can apply a force on the tab 444 to break it off, thus applying a force to the piston 407 to break the stiction between the reservoir chamber 401 and the reservoir chamber piston 407. In other embodiments, the packaging that holds the disposable part 415 can include or be attached to the tab 444 (which can be a breakaway tab or a fixed tab). When the packaging is opened and/or the disposable part 415 is removed by the user, the tab 444 can simultaneously be broken or removed, thus applying a force on the piston 407 to break the stiction. Similar to device 300, the bolus chamber 403 can be only partially filled before use, thereby providing room for the bolus chamber piston 409 to move backwards to break the stiction thereof when the stiction is broken on the piston 407.

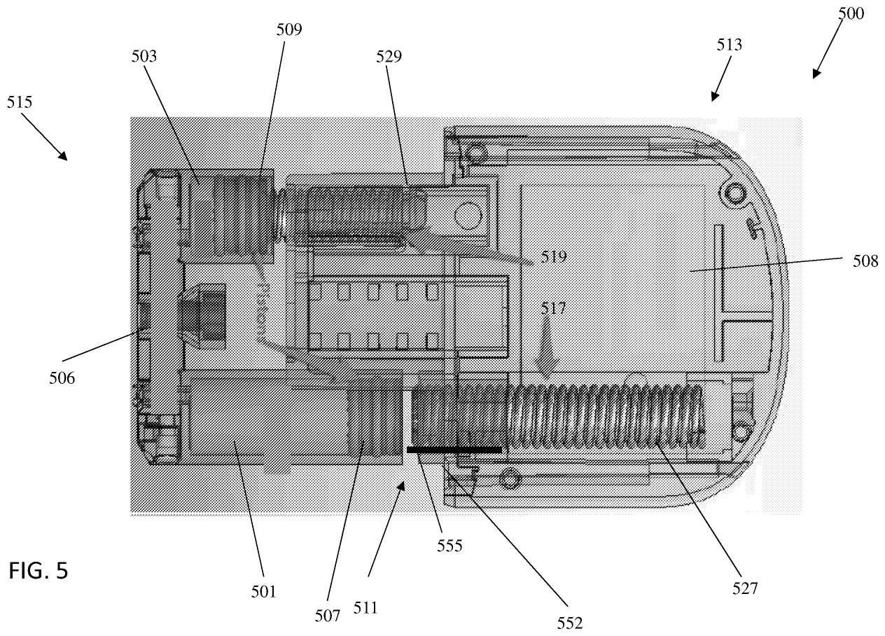

FIG. 5 illustrates an embodiment of a transdermal drug delivery system 500 that includes a stiction breaking mechanism 511. Similar to other embodiments described herein, the device 500 includes a reusable part 513, a reservoir shaft 527, a reservoir spring 517, a bolus shaft 529, and a bolus spring 519. The disposable part 515 includes a reservoir chamber 501, a reservoir chamber piston 507, a bolus chamber 503, a bolus chamber piston 509, and a control valve 506. The reusable part includes a piston stiction breaking element in the form of a stationary piston engagement rod 555 that protrudes slightly from the housing 552 adjacent to the reservoir spring 517 and reservoir shaft 527 and in-line with at least a portion of the reservoir chamber piston 507. When the reusable part 513 and disposable part 515 are engaged together, the piston engagement rod 555 provides a force on the reservoir chamber piston 507 to break the stiction between the reservoir chamber piston 507 and the reservoir chamber 501. Similar to other embodiments, the force can also break the stiction of bolus chamber piston 509.

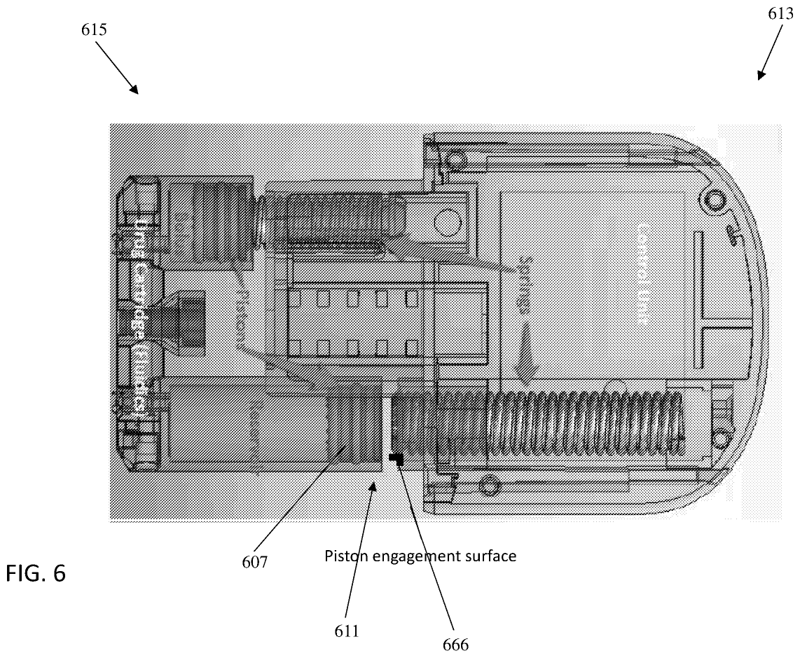

FIG. 6 illustrates another embodiment of a piston stiction breaking element 611 on a reusable part 613. The piston stiction breaking element 611 is similar to element 511, but is in the form of a small tab 666 rather than a rod. The tab 666 exerts the stiction breaking force on the piston 607 when the reusable part 613 and the disposable part 615 are engaged together.

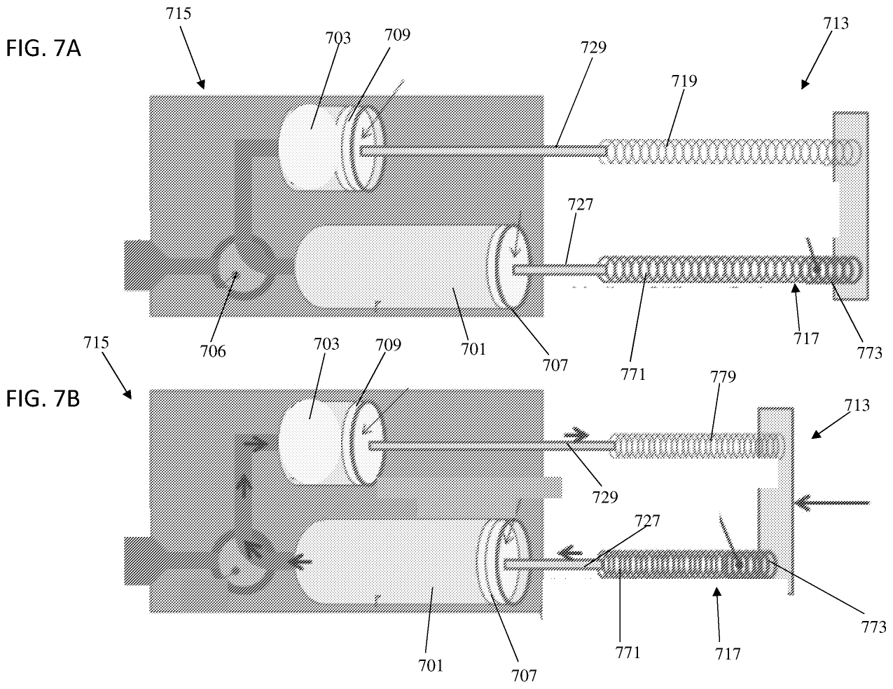

FIGS. 7A and 7B schematically illustrate another transdermal delivery device 700 with a stiction breaking mechanism 711. Similar to other embodiments described herein, the device 700 includes a reusable part 713 including a reservoir shaft 727, a reservoir spring 717, a bolus shaft 729, and a bolus spring 719. The disposable part 715 includes a reservoir chamber 701, a reservoir chamber piston 707, a bolus chamber 703, a bolus chamber piston 709, and a control valve 706. Further, the stiction breaking mechanism 711 is built into the springs 717, 719. That is, the reservoir spring 717 can include a medium stiffness portion 771 at the distal end thereof and a high stiffness portion 773 at the proximal end thereof (i.e., having a higher stiffness than the medium stiffness portion 771). In some embodiments, the springs can also be arranged in the opposite orientation with the high stiffness portion 773 at the distal end and the medium stiffness portion 771 at the proximal end. Further, the bolus spring 419 can have a lower stiffness than the medium stiffness portion 771. For example, the high stiffness portion 773 can have a stiffness of 15-25N, the medium stiffness portion 771 can have a stiffness of 5-10N, and the bolus spring 419 can have a stiffness of 2-6N. As shown in the transition from FIG. 7A to FIG. 7B, the reservoir spring 717 can be compressed when the reusable part 713 and disposable part 715 are engaged together. As a result of the high stiffness portion 773, the initial engagement can cause the reservoir spring 717 to exert a relatively large force through the shaft 727 to break the stiction between the reservoir chamber piston 707 and reservoir chamber 701. Breaking the stiction in the reservoir chamber 701 causes a small amount of fluid to travel out of the reservoir chamber 701 and into the bolus chamber 703. The fluid movement into the bolus chamber 703 can exert a force on the piston 709, causing the piston 709 to move backwards to break the stiction of the piston 709 as well. In some embodiments, the two portions 771, 773 can be arranged in parallel to one another rather than in series. The medium stiffness portion 771 can travel the entire length of the reservoir 701 while the high stiffness portion 773 can be shorter and engage only during stiction break.

Any of the piston stiction breaking mechanisms described herein can be applied directly to either or both of the pistons of the transdermal drug delivery device. Although the illustrated examples break the stiction through direct contact with the piston on the drug reservoir, the same concepts and structures could instead be applied to the bolus/dosing chamber piston to directly contact that piston to break the stiction there first with the fluid then exerting a force on the piston in the reservoir chamber to break the stiction on that piston.

Although described as including a disposable part and a reusable part, it is to be understood that the two-part designs described herein are applicable to systems where both parts are reusable or both parts are disposable. For example, the disposable part can be synonymous with a cartridge (disposable or reusable) while the reusable part can be synonymous with a control unit (disposable or reusable).

Any of the features described with respect to any one of the embodiments herein can be combined with or used to replace any of the features described with respect to any other embodiment.

The systems described herein can include a transdermal membrane that contacts the wearer's skin. A drug or other bioactive agent and solvent solution can be delivered in a controlled amount to the transdermal membrane. The transdermal membrane can be configured to minimize permeation of the solvent solution while permitting diffusion of the drug or other bioactive agent across the membrane and into contact with the skin. The solvent solution can be removed through a vapor permeable membrane. The systems described herein can efficiently deliver substantially all of the drug or other bioactive agent across the transdermal membrane into contact with the wearer's skin. The removed solvent can be collected in a solvent removal element. An example of a solvent removal element that can be used in the transdermal drug delivery devices described herein is disclosed in U.S. Pat. No. 8,673,346, the disclosure of which is incorporated by reference in its entirety.

The drug delivery profile can correspond to a circadian rhythm or a bio-synchronous pattern of a patient using the transdermal drug delivery device. Examples of circadian rhythm or a bio-synchronous drug delivery profile that can be used with the devices described herein are disclosed in US 2015-0283367 and U.S. Pat. No. 8,741,336, the disclosures of each of which are incorporated by reference in its entirety.

The transdermal membrane may be any appropriate material(s) or have any appropriate characteristics that can transfer the bioactive agent across the membrane. A membrane may be hydrophilic or hydrophobic. A membrane may have pores, such as from 0.010-0.01 μm (e.g., from 0.02 μm-0.05 μm, etc.). A membrane may have porosity over 20%-60% (e.g., from 30%-50%, from 45% to 50%, etc.). In a particular example a polypropylene such as Celgard 2400 polypropylene (e.g., with a thickness around 25 μm such as between 1 μm and 100 μm, with a pore size around 0.043 such as from 0.005 to 0.2 μm, etc. may be used). A material may be chosen based on the bioactive agent, length of treatment, etc.

The composition of the solvent can also be designed and selected to optimize the diffusion of the drug or bioactive agent across the transdermal membrane. The composition of the solvent can also be chosen in combination with the transdermal membrane to achieve the desired drug or bioactive agent delivery rate.

The devices described herein can include a solvent removal element, such an absorbent to receive and hold the solvent. The solvent removal element can be part of the disposable part or cartridge. An absorbent for use with a transdermal patch as described herein may be an absorbent gel, blotting paper, paper, other polymer, silica gel or other material that readily soaks up or holds a fluid media such as a solvent liquid or vapor. An absorbent generally behaves as a physical sponge. An absorbent may be any structure or shape such as a single piece or a plurality of pieces. An absorbent may be an amorphous material or a formed material, and may be a block, a layer, a sheet, a plurality of sheets, a plurality of particles and so on. A desiccant may be used instead or in addition to the absorbent.

A solvent for a bioactive agent may include a single component or multiple components such as alcohol, water, or another solvent that readily vaporizes. One or more than one component may vaporize and be absorbed by an absorbent. A vapor/gas permeable membrane may contain discrete pores that extend from one side of the membrane to the other side and allow gas to flow through.

In some embodiments the solvent solution includes water, alcohol, and a drug or bioactive agent. In some embodiments the alcohol can be one or more of isopropanol, ethanol, and methanol. The solvent solution can also include one or more of a: surfactant, excipient, or other component intended to enhance permeation or decrease skin sensitivity or skin reaction.

The solvent solution can have a ratio of water to alcohol of about 40:60 to about 60:40. The solvent solution can have a ratio of water to alcohol of about 45:55 to about 55:45. The solvent solution can have a ratio of water to alcohol of about 46:54 to about 54:46. The solvent solution can have a ratio of water to alcohol of about 47:53 to about 53:47. The solvent solution can have a ratio of water to alcohol of about 48:52 to about 52:48. The solvent solution can have a ratio of water to alcohol of about 49:51 to about 51:49.

A variety of different drugs or bioactive agents can be used with the systems described herein. In some embodiments the bioactive agent includes nicotine. For example, nicotine can be present in the solvent solution from about 0.5% to about 20% by volume. In some embodiments nicotine can be present in the solvent solution from about 0.5% to about 10% by volume. In some embodiments nicotine can be present in the solvent solution from about 0.5% to about 5% by volume. In some embodiments nicotine can be present in the solvent solution from about 0.5% to about 3% by volume.

In one example the bioactive agent is nicotine. Other examples of bioactive agents include: Acamprosate, Acetaminophen, Acetaminophen+Oxycodone, Alevicyn SG, Alfentanil, Allopurinol, Almotriptan, Alprazolam, Alprazolam XR, Amitriptylinem, Amoxapine, Apomorphine, Aripiprazole, Armodafinil, Asenapine maleate, Atomoxetine, Azelastine HCL, Baclofen, Benzbromarone, Benzydamine, Brexpiprazole, Budesonide, Bupivacaine, Buprenorphine, Buprenorphine+Nalaxone, Bupropion, Bupropion Hydrobromide, Bupropion Hydrochloride, Bupropion SR, Bupropion XR, Buspirone, Cabergoline, Capsaicin, Carbamazepine CR, Carbamazepine XR, Carbidopa+Levodopa Er, Carisprodol, Celecoxib, Citalopram, Clobazam, Clonazepam, Clonidine Patch, Clonidine SR, Clopidogrel, Colchicine, Cyclobenzaprine ER, Cyclobenzaprine PO, Dalteparin sodium, Desvenlafaxine, Desvenlafaxine ER, Dexamfetamine, Dexmethylphenidate Hcl, Dexmethylphenidate Hcl LA, Diazepam, Diclofenacm, Diclofenac Gel, Diclofenac IR, Diclofenac IV, Diclofenac Potassium IR, Diclofenac Potassium XR, Diclofenac Transdermal, Disulfiram, Divalproex Sodium, Dolasetron Mesilate, Doxepin, Dronabinol, Droxidopa, Duloxetine, Eletriptan, Entacapone, Escitalopram oxalate, Eslicarbazepine Acetate, Esomeprazole/naproxen, Estradiol, Estrogen, Eszopiclone, Ethosuximide, Etodolac, Ezogabine, Febuxostat, Felbamate, Fenbufen, Fentanyl Citrate, Fentanyl Oral, Fentanyl Patch, Fentanyl SL, Flunisolide, Fluorouracil, Fluoxetine, Fluticasone propionate, Fluvoxamine Cr, Formoterol, Fosphenytoin, Frovatriptan, Gabapentin, Gabapentin ER, Granisetron ER, Guanfacine, Hydrocodone Bitartrate CR, Hydrocodone+Acetaminophen, hydrocortisone, Hydromorphone Hcl, Hydroxyzine, Hypericum Extract, Ibuprofen, Indometacin, Ketorolac, Lacosamide, Lamotrigine, Lamotrigine CDT, Lamotrigine ODT, Lamotrigine XR, Levetiracetam, Levetiracetam IR, Levetiracetam XR, Levomilnacipran, Levosalbutamol, Lidocaine Patch, Lidocaine/Tetracaine, Lisdexamfetamine, Lithium Carbonate, Lorazepam, Lorcaserin, Hydrochloride, Losartan, Loxapine, Meclizine, Meloxicam, Metaxalone, Methylphenidate, Methylphenidate Hydrochloride, Methylphenidate LA, Methylphenidate MR, Methylphenidate Patch, Milnacipran, Mirtazapine, Modafinil, Morphine, Morphine CR, Morphine ER, Nabilone, Nadolol, Naltrexone, Naproxen, Naratriptan, Nedocromil, Nefazodone, Nitroglycerin, Nitroglycerin Ointment, Olanzapine, Olanzapine IM, Olanzapine LA, Ondansetron, Ondansetron ODFS, Ondansetron ODT, Orlistat, Oxaprozin, Oxcarbazepine, Oxcarbazepine ER, Oxybutynin, Oxybutynin Gel, Oxycodone, Oxycodone+Acetominophen, Oxycodone Hydrochloride, Oxycodone IR, Oxymorphone, Oxymorphone ER, Palonosetro, Pamidronate, Paroxetine, Paroxetine Mesylate, Perampanel, Phentermine+Topiramate, Phentermine Hydrochloride, Phentolamine Mesylate, Pramipexole, Pramipexole-Er, Prasugrel, Prazepam, Prednisone, Pregabalin, Promethazine, Propofol, Quetiapine, Quetiapine Fumarate, Quetiapine Fumarate XR, Ramelteon, Rasagiline Mesylate, Remifentanil, Risperidone, Rivastigmine Tartrate, Rizatriptan, Ropinirole, Ropinirole XL, Ropivacaine, Rotigotine, Rufinamide, Salbutamol, Scopolamine, Selegiline, Selegiline ODT, Selegiline Transdermal, Sertraline, Sodium Oxybate, Strontium, Sufentanil-Ent, Sumatriptan Autoinjector, Sumatriptan Needle-free, Sumatriptan Succinate, Suvorexant, Tapentadol, Tapentadol ER, Tasimelteon, Temazepam, Testosterone, Tetracaine+Lidocaine, Theophylline, Tiagabine, Tiotropium, Tirofiban HcL, Tolcapone, Topiramate, Topiramate XR, Tramadol, Tramadol+Acetaminophen, Tramadol ER, Trazodone Cr, Triazolam, Trimipramine Maleate, Valproate Semisodium ER, Valproate Sodium, Venlafaxine, Venlafaxine ER, Vigabatrin, Vilazodone, Vortioxetine, Zaleplon, Zileuton, Ziprasidone, Zolmitriptan Oral, Zolmitriptan ZMT, Zolpidem, Zolpidem Spray, Zolpidem Tartrate CR, Zolpidem Tartrate Low dose SL, Zolpidem Tartrate SL, norethisterone acetate (NETA), enapril, ethinyl estradiol, insulin, memantine, methamphetamine, norelgestromine, pergolide, Ramipril, tecrine, timolol, tolterodine and Zonisamide.

Other aspects of delivery systems, such as transdermal delivery systems, are described in US Publication No. 2016/0220798, titled “Drug Delivery Methods and Systems”, which is incorporated by reference herein in its entirety.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A formulation delivery system comprising:
a cartridge comprising:
a reservoir chamber configured to hold a formulation therein;
a bolus chamber in fluid communication with the reservoir chamber through a fluid communication pathway, the bolus chamber configured to hold a portion of the formulation from the reservoir chamber;
a reservoir chamber piston configured to move within the reservoir chamber to expel the formulation from the reservoir chamber into the bolus chamber; and
a bolus chamber piston configured to move within the bolus chamber to expel the formulation from the bolus chamber to a patient;
a control unit configured to engage with the cartridge, the control unit including a control configured to activate the reservoir chamber piston or the bolus chamber piston; and
a piston stiction breaking element configured to break a first stiction between the reservoir chamber piston and the reservoir chamber or between the bolus chamber piston and the bolus chamber.

2. The formulation delivery system of claim 1, wherein the piston stiction breaking element is configured such that, as the cartridge and the control unit are engaged, the piston stiction breaking element applies a force to the reservoir chamber piston or the bolus chamber piston to break the first stiction.

3. The formulation delivery system of claim 2, wherein the fluid communication pathway is configured such that, when the piston stiction breaking element applies the force to the reservoir chamber piston or the bolus chamber piston to break the first stiction, the force is transferred through the formulation to the other of the reservoir chamber piston or the bolus chamber piston to break a second stiction.

4. The formulation delivery system of claim 3, wherein the reservoir chamber piston and the bolus chamber piston both move by a distance d when the first and second stictions are broken.

5. The formulation delivery system of claim 4, wherein the distance d is between approximately 0.5 mm and 2.5 mm.

6. The formulation delivery system of claim 1, wherein the control unit further includes a first shaft and first spring, the first spring configured to provide force to the first shaft to move the reservoir chamber piston or the bolus chamber piston.

7. The formulation delivery system of claim 6, wherein the piston stiction breaking element is an extension on the first shaft, the extension configured such that it pushes the reservoir chamber piston or bolus chamber piston as the cartridge and control unit are engaged to break the first stiction.

8. The formulation delivery system of claim 7, wherein the control unit includes a stop therein, the stop configured to engage with the first spring or the first shaft as the control unit and cartridge are engaged to prevent movement of the first spring and the first shaft distally and to allow the extension to push the reservoir chamber piston or the bolus chamber piston.

9. The formulation delivery system of claim 6, wherein the extension is configured such that the extension pushes the reservoir chamber piston.

10. The formulation delivery system of claim 6, further comprising a second shaft and a second spring in the control unit, the second shaft configured to provide force to the second shaft to move the other of the reservoir chamber piston or the bolus chamber piston.

11. The formulation delivery system of claim 6, wherein the first spring is configured to provide approximately 15N of force or less to break the first stiction.

12. The formulation delivery system of claim 1, wherein the piston stiction breaking element is a tab configured to engage with the reservoir chamber piston or the bolus chamber piston.

13. The formulation delivery system of claim 12, wherein the tab is a breakaway tab.

14. The formulation delivery system of claim 13, wherein the breakaway tab is configured such that a force applied to break off the breakaway tab also breaks the first stiction.

15. The formulation delivery system of claim 12, wherein the tab is attached to or part of a packaging of the cartridge, and wherein removing the packaging causes the tab to break the first stiction.

16. The formulation delivery system of claim 1, wherein the piston stiction breaking element is part of a spring system, the spring system including a first spring having greater stiffness than a second spring, the first spring configured to provide force to the bolus chamber piston or the reservoir chamber piston to break the first stiction.

17. The formulation delivery system of claim 1, wherein the first spring includes a first portion and a second portion, the first portion having a higher stiffness than the second portion.

18. The formulation delivery system of claim 1, wherein the piston stiction breaking element is a rod extending from the cartridge, the rod configured to push the reservoir chamber piston or the bolus chamber piston as the cartridge and the control unit are engaged.

19. The formulation delivery system of claim 1, wherein the drug delivery system is a transdermal drug delivery system, the system further comprising a transdermal membrane in fluid connection with the bolus chamber.

20. The formulation delivery system of claim 1, further comprising a control valve with a first position forming the first fluid communication pathway between the bolus chamber and the reservoir chamber and a second position forming a second fluid communication pathway between the bolus chamber and a transdermal membrane.

21. The formulation delivery system of claim 1, wherein a total force required to start movement of the bolus chamber piston or reservoir chamber piston is between approximately 2N and 15N.

22. A formulation delivery system comprising:
a cartridge comprising:
a reservoir chamber configured to hold a formulation therein; and
a reservoir chamber piston configured to move within the reservoir chamber to expel the formulation from the reservoir chamber;
a control unit configured to engage with the cartridge, the control unit comprising:
a shaft;
a spring, the spring configured to provide force to the shaft to move the reservoir chamber piston; and
a control configured to activate the spring; and
a piston stiction breaking element configured to break a first stiction between the reservoir chamber piston and the reservoir chamber.

23. The formulation delivery system of claim 22, further comprising a bolus chamber in fluid communication with the reservoir chamber through a fluid communication pathway, the bolus chamber configured to hold a portion of the formulation from the reservoir chamber.

24. The formulation delivery system of claim 23, further comprising a bolus chamber piston configured to move within the bolus chamber to expel the formulation from the bolus chamber to a patient.

26. The formulation delivery system of claim 24, wherein the fluid communication pathway is configured such that, when the piston stiction breaking element applies the force to the reservoir chamber piston to break the stiction, the force is transferred through the formulation to the bolus chamber piston to break a second stiction between the bolus chamber piston and the bolus chamber.

27. The formulation delivery system of claim 26, wherein the reservoir chamber piston and the bolus chamber piston both move by a distance d when the first and second stictions are broken.

28. The formulation delivery system of claim 27, wherein the distance d is between approximately 0.5 mm and 2.5 mm.

29. The formulation delivery system of claim 22, wherein the piston stiction breaking element is configured such that, as the cartridge and the control unit are engaged, the piston stiction breaking element applies a force to the reservoir chamber piston to break the first stiction.

30. The formulation delivery system of claim 22, wherein the piston stiction breaking element is an extension on the shaft, the extension configured such that it pushes the reservoir chamber piston as the cartridge and control unit are engaged to break the first stiction.

31. The formulation delivery system of claim 30, wherein the control unit includes a stop therein, the stop configured to engage with the spring or the shaft as the control unit and cartridge are engaged to prevent movement of the spring and the shaft distally and to allow the extension to push the reservoir chamber piston.

32. The formulation delivery system of claim 22, wherein the spring is configured to provide approximately 15N or less of force to break the first stiction.

33. The formulation delivery system of claim 22, wherein the piston stiction breaking element is a tab configured to engage with the reservoir chamber piston.

34. The formulation delivery system of claim 33, wherein the tab is a breakaway tab.

35. The formulation delivery system of claim 34, wherein the breakaway tab is configured such that a force applied to break off the breakaway tab also breaks the first stiction.

36. The formulation delivery system of claim 33, wherein the tab is attached to or part of a packaging of the cartridge, and wherein removing the packaging causes the tab to break the first stiction.

37. The formulation delivery system of claim 22, wherein the piston stiction breaking element is part of the spring, wherein the spring includes a first portion and a second portion, the first portion having a higher stiffness than the second portion.

38. The formulation delivery system of claim 22, wherein the piston stiction breaking element is a rod extending from the cartridge, the rod configured to push the reservoir chamber piston as the cartridge and the control unit are engaged.

39. The formulation delivery system of claim 22, wherein the drug delivery system is a transdermal drug delivery system, the system further comprising a transdermal membrane in fluid connection with the reservoir.

40. The formulation delivery system of claim 22, wherein a total force required to start movement of the reservoir chamber piston is between approximately 2N and 15N.

41. A method of using a transdermal drug delivery system comprising:
engaging a cartridge of the transdermal drug delivery system with a control unit of the transdermal drug delivery system, wherein the cartridge includes a reservoir chamber with a formulation therein, a bolus chamber with a formulation therein, a reservoir chamber piston, and a bolus chamber piston;
applying force to lock the cartridge and control unit together, wherein the application of force activates a stiction breaking mechanism to break a first stiction between the reservoir chamber piston and the reservoir chamber or a second stiction between the bolus chamber piston and the bolus chamber; and
allowing the formulation to be delivered to a patient.

42. The method of claim 41, wherein the force applied to the lock the cartridge and control unit together is between approximately 12N and 35N.

43. The method of claim 41, wherein the application of force breaks the first stiction and the second stiction.

44. A method of using a transdermal drug delivery system comprising:
engaging a cartridge of the transdermal drug delivery system with a control unit of the transdermal drug delivery system, wherein the cartridge includes a reservoir chamber with a formulation therein, a bolus chamber with a formulation therein, a reservoir chamber piston, and a bolus chamber piston;
pulling a tab to break a first stiction between the reservoir chamber piston and the reservoir chamber or a second stiction between the bolus chamber piston and the bolus chamber;
applying force to lock the cartridge and control unit together; and
allowing the formulation to be delivered to a patient.

45. The method of claim 44, wherein pulling the tab breaks the first stiction and the second stiction.

46. The method of claim 44, wherein pulling the tab comprises unwrapping the cartridge from a packaging, the unwrapping pulling the tab.

47. The method of claim 46, wherein the tab is fixed to the packaging.
